(19) United States
(12) Patent Application Publication
(10) Pub. No.: US 2021/0353512 A1
(43) Pub. Date: Nov. 18, 2021

(54) NOVEL ENCAPSULATED COSMETIC COMPOSITIONS AND USES THEREOF

(71) Applicant: ELC Management LLC, Melville, NY (US)

(72) Inventor: Wilson A. Lee, Hauppauge, NY (US)

(21) Appl. No.: 17/319,076

(22) Filed: May 12, 2021

Related U.S. Application Data
(60) Provisional application No. 63/025,759, filed on May 15, 2020.

(51) Int. Cl. A61K 8/02; A61K 8/11; A61Q 17/04; A61Q 19/00; A61Q 1/02; A61K 8/29; A61K 8/27

(52) U.S. Cl. CPC A61K 8/0275; A61K 8/11; A61Q 17/04; A61K 2800/43; A61Q 1/02; A61K 8/29; A61K 8/27; A61Q 19/00

(57) ABSTRACT

Embodiments herein provide a cosmetic or dermatologic composition comprising a plurality of multiparticulated particles, wherein the multiparticulated particles comprise a plurality of coated active ingredient particles, each coated particle comprising at least one active ingredient having a core and a coating thereon. The multiparticulated particles further comprise a plurality of uncoated active ingredient particles, the uncoated particles comprising at least one active ingredient having a core. In further embodiments, the coated particles and the uncoated particles have the same or different active ingredient cores. The present invention further provides a method of protecting skin or hair by applying the cosmetic composition according to the invention.

### FIELD

The present invention generally relates to novel compositions for coating substrates and/or surfaces. Specifically, the present invention relates to novel cosmetic or dermatologic compositions to protect skin, particularly in applications directed to sun care and make-up.

### BACKGROUND

The human body is very sensitive to sunlight, artificial light, and pollutants. Prolonged exposure to irritants, pollutants and ultraviolet radiation affects the layers of human skin, resulting in skin damage, redness, eruptions, skin burn, and the like. With increased awareness on the damage caused by such agents on the skin, and the technological developments, many cosmetic and dermatologic products have become commercially available with attributes to protect human skin. Notably, many commercially available products are not effective in protecting human body against the damage caused by the harmful agents. For example, the effectiveness of sunscreen products is indicated by the sun protection factors (SPF). In general, sunscreen compositions are formulated as creams, lotions or oils containing an active agent that blocks passage of ultraviolet radiation, thereby preventing penetration of such radiation into the skin. However, many commercially available formulations are not stable due to agglomeration and uneven distribution and non-uniform pore size of the active agents.

Agglomeration results in undesirable formulations that have transparent particles, which are visible to the naked eye. Agglomeration also causes leaching or swirling of the active agent, leakage of color and thereby, compromises stability and effectiveness of the sun protection product. In case of the sunscreen composition, agglomeration and leakage and in some instances, increased free radical generation compromises the effectiveness of the product, allowing the ultraviolet radiation to penetrate the human skin. In other instances, for example, in a make-up composition, the increased free radical generation, and agglomeration results in leakage of color and excipients present in the composition, thereby, affecting the shelf life of the composition.

Similarly, many environmental pollutants, micro-bacterial and viral particles are transmitted from surface to surface via contact. Protecting surfaces from the bacterial or viral particles is an objective of multiple research studies and many coating applications are being utilized for this purpose; however, the primary challenge is preventing the entry of pollutants, bacterial or viral particles, particularly in view of the very small particle size of such particles while facilitating airflow through the surface and/or substrate.

It is therefore an object of the present invention to provide novel compositions for coating substrates and/or surfaces. It is also an object of the present invention to provide novel cosmetic or dermatologic preparations with coated active ingredients incorporated into cosmetic and dermatologic formulations, a process of producing such compositions and uses thereof.

### SUMMARY

Embodiments herein provide a cosmetic composition comprising a plurality of multiparticulated particles, wherein the multiparticulated particles comprise a plurality of coated active ingredient particles, each coated particle comprising at least one active ingredient having a core and a coating thereon. The multiparticulated particles further comprise a plurality of uncoated active ingredient particles, the uncoated particles comprising at least one active ingredient having a core. In further embodiments, the coated particles and the uncoated particles have the same or different active ingredient cores. The active ingredient is a cosmetic agent, peptide, DNA, vitamin, organic acid, pigment, or protein. In specific embodiments, the particles are coated with a coating composition comprising at least one polyacrylates copolymer or a complex thereof.

The present invention further provides a method of protecting the human skin or hair by applying the cosmetic composition described herein.

### BRIEF DESCRIPTION OF THE DRAWINGS

None.

### DETAILED DESCRIPTION

To facilitate an understanding of this invention, several terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

As used herein, a “cosmetic agent” means agents or active ingredients suitable for topical application on mammalian keratinous tissue. The cosmetic agent may be a substance that aids in the cleansing or enhancement or protection of a subject's skin or the appearance (e.g., color, texture, look, feel, etc.) or odor of the subject's skin, body, or hair. The cosmetic agent may change the underlying structure of the skin or hair.

As used herein, the terms “prevent” and “preventing” include the prevention of the recurrence, spread, or onset of a skin or hair condition. It is not intended that the present invention be limited to complete prevention.

A “subject” refers to any mammal, preferably a human.

As used herein, the term “topical” refers to the administration of an agent or agents (e.g., cosmetic, vitamin, etc.) on the skin.

The term “T_{g}” refers to the glass transition temperature.

The term “zeta potential” refers to a measure of potential difference in the charge that exists between a dispersion medium, i.e., coating and the stationary layer of a particle. In the case of skincare or sun care composition, the zeta potential measures the amount of free radicals generated before and after the active ingredient(s) is illuminated by UV light.

Except in operating and comparative examples, or where otherwise explicitly indicated, all numbers in this description indicating amounts or ratios of substrate or conditions of reaction, physical properties of substrates, and/or use are to be understood as modified by the word “about”. All amounts are presented as percentages by weight of the final composition unless otherwise specified.

### Coating Composition

It is an aspect of the present invention to provide a novel coating composition comprising at least one polyacrylates copolymer or a complex thereof for the purpose of coating cosmetic active ingredients and/or non-cosmetic substrate materials for commercial, industrial, and clinical uses.

Acrylates copolymers are film-forming polymers formed of two or more monomers of acrylic acid, methacrylic acid or one of the simple esters. The chemical structure of acrylates copolymer is provided below.

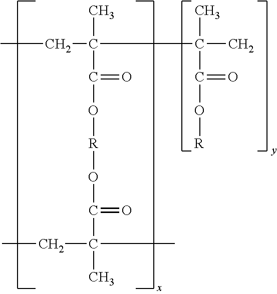

Polyacrylates copolymer, also known as poly(2-propenamide) polymer is formed from acrylamide subunits. It may exist in a cross-linked form or as a linear chain structure. It is highly water absorbent and forms a hydrogel when it interacts with water. Polyacrylates comprising amide groups may be hydrolyzed, forming carboxyl groups in the side chains of the polyacrylates polymer backbone. Primary amines are formed inside chains of the polymer backbone via a Hofmann Rearrangement reaction. Acrylamide monomers may also be copolymerized with a number of different monomers, thereby, producing acrylamide or acrylates copolymers comprising various reactive chemical groups. Exemplary structures of polyacrylates are provided below.

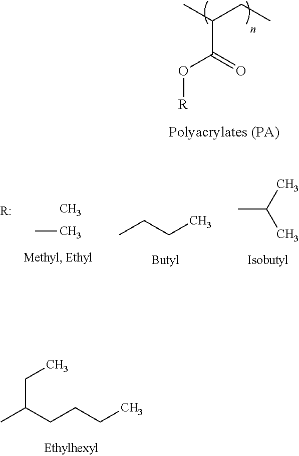

Polyacrylates copolymers are soft, tough, and rubbery. They are also highly transparent with good impact toughness and elasticity, heat resistance, weatherability and ozone resistance due to the lack of double bonds in the backbone structure. Polyacrylates copolymer according to the present invention comprises poly(methyl acrylate), poly(ethyl acrylate), butyl, benzyl, 4-cholorophenyl, 2-cyanomethyl, cyclohexyl, ethyl, hexyl, isobutyl, propyl, sec-butyl, tert-butyl, 2-ethylhexyl acrylate, methacrylates, vinyl acrylates, ammonium acrylates, styrenes, acrylamide, acrylonitrile among others. Methacrylate, styrene and acrylonitrile polymers increase the cohesive strength (hardness) and reduce or eliminate the tacky feel on the skin upon drying.

According to one aspect, the present invention relates to a coating composition, wherein the coating composition comprises at least one polyacrylates copolymer that exists as a linear polyacrylates copolymer or as a complex of acrylates with more than one chemical compound, such as, without limiting, acrylic acid, or salt.

According to the present invention, the at least one polyacrylates copolymer or a complex thereof has a molecular weight of about 2000 to about 1000000 g/mole. In one embodiment, the polyacrylates copolymer has an average molecular weight from about 50,000 to 150,000 g/mole, preferably ranging from about 84,000 to about 125,000 g/mole and T_{g} ranging from about −40 to 60 degree Celsius, and preferably about −30 to about 30 degree Celsius. In one embodiment, the polyacrylates copolymer or a complex thereof, employed in the coating composition is present in amounts ranging from about 0.1% to about 95% by weight, preferably from about 40% to about 90% by weight based on the total weight of the coating composition. The choice of the polyacrylates copolymer or the polyacrylates copolymer complex in the coating composition depends upon the substrate that is being coated. Another factor that influences the choice of the polyacrylates copolymer or the complex is the desired porosity of the final coating composition and the desired physical characteristics of the coating composition. The coating compositions according to the present invention are aqueous and may typically comprise from about 50% to about 60% of water by weight of the total coating composition. However, the coating composition may not be sprayable or may be very thin for commercial or industrial handling. Further, the coating composition may be non-porous or in some instances, the pore size may be too large. In order to achieve the desired pore size and flexibility, excipients and carriers such as butylene glycol, propanediol, glycerine, or any combination thereof are employed in the coating composition, at amounts ranging from about 0.1% to 5% by the total weight of the coating composition. In specific embodiments, the desired pore size may range from about 0.1 nm to 5 μm, including subranges within.

Moreover, for commercial purposes, increased adhesion of the coating composition to the substrate is desired. This may be achieved by ensuring that the surface tension of the dried substrate is within 10 mN/m (milli-newtons per meter) of the surface tension of the substrate. In general, increasing the level of one or more of butylene glycol, propanediol, and glycerine in the coating composition will lower the surface tension of the coating composition. Advantageously, the surface tension of the coating composition can be adjusted, as needed, by the use of 0.1% to 5% butylene glycol, propanediol, glycerine, or any combination thereof. Thus, the benefits of sprayability, flexibility, comfort, and adhesion are achieved by adjusting the total concentration of any combination of these ingredients to be about 0.1% to 5% by weight of the total coating composition.

Yet another factor that would determine the choice of the polyacrylates copolymer or the polyacrylates copolymer complex in the coating composition is the Young's modulus of the polyacrylates copolymer or the complex thereof as well as the combined Young's modulus of the copolymers. The Young's modulus of the copolymers would affect the handling characteristics of the coating composition, including stiffness or flexibility as well as the adhesion of the copolymers to the substrate. Notably, Young's modulus of the copolymers will also have an effect on the porosity since an increase in the stiffness would increase the porosity of the coating composition. As described herein, for the non-cosmetic applications utilizing the coating composition of the present invention, it is desirable to control the pore size to allow airflow yet prevent entry of pollutants, bacterial or viral particles. In cosmetic applications, it is desirable to prevent the leakage of free radicals generated within the active agents as well as the leakage of color from pigments from the active ingredient into the cosmetic composition.

In some embodiments, polyacrylates copolymer or the complex thereof employs Young's modulus in the range of about 0.001 pascal units to about 100 Gpascal units. In some embodiments, the Young's modulus is in the range of about 0.001 Gpascal units to about 20 Gpascal units. In some embodiments, the Young's modulus in the range of about 0.001 Gpascal units to about 10 Gpascal units. In some other embodiments, the combined polyacrylates copolymer or the complex thereof employs Young's modulus in the range of about 0.001 pascal units to about 100 Gpascal units.

As noted herein, in some embodiments, the substrate may be cosmetic active ingredients. In other embodiments, the substrate may be a non-cosmetic material, including plastic, metal, paper, cloth/textile, paper, drug, pharmaceutical products, among others. In further embodiments, the substrate may also be any blood-contacting material conventionally used for hospital or clinical use, such as storage bags, pacemaker leads, vascular grafts, hospital use devices. In some embodiments, the substrate may be modified on its surface via for example, without limiting, polyurethane, polydimethylsiloxane, polytetrafluoroethylene, polyvinylchloride, Dacron™ or a composite made therefrom. Moreover, the substrate may also be an inorganic or metallic base material with or without suitable reactive groups, e.g., ceramic, quartz, or metals, such as silicon or gold, or other polymeric or non-polymeric substrate materials.

In one embodiment, at least one polyacrylates copolymer is utilized for coating. In another embodiment, a polymer complex comprising polyacrylates copolymers is utilized. Examples of suitable polymers include ethylenically unsaturated sulfur acid functional monomers including sulfoethyl (meth)acrylate, sulfopropyl (meth)acrylate, styrene sulfonic acid, vinyl sulfonic acid, and 2-(meth)acrylamido-2-methyl propanesulfonic acid, and salts thereof, with 2-(meth)acrylamido-2-methyl propanesulfonic acid, sulfoethyl methacrylate, (meth)acrylamide, poly (methyl acrylate), poly(ethyl acrylate), butyl, benzyl, 4-cholorophenyl, 2-cyanomethyl, cyclohexyl, ethyl, hexyl, isobutyl, propy, sec-butyl, tert-butyl, 2-ethylhexyl acrylate, methacrylates, vinyl acrylates, ammonium acrylates, styrenes, acrylamide, acrylonitrile. In addition to the above polymers, amphoteric polymers may also be utilized. The polymers are initially dispersed in water prior to a spraying step. After the spraying step, the substrate is dried to eliminate moisture.

In one embodiment, the thickness of the coating present on the surface of the substrate can be varied between 0.1 nm to 100 mm. In some embodiments, the thickness of the coating is in the range of about 0.5 nm and 50 mm.

In certain embodiments, the polymer complex, comprising polyacrylates copolymer may be present in amounts ranging from about 1% to about 60% by weight of the coating composition. In some embodiments, the polymer complex comprising polyacrylates has less than 50% moisture content after drying. In preferred embodiments, it has less than 10%, 5%, 1%, or 0.1% moisture content after drying.

In some embodiments, the ratio of the substrate to the coating composition ranges from about 1:1 to about 99:1. In some embodiments, the ratio is about 1:1 to about 50:99. In some embodiments, the ratio is about 1:1 to about 50:1. In some embodiments, the ratio of the substrate to the coating ranges from about 1:1 to about 1:99. In some embodiments, the ratio is about 1:1 to about 1:50. In some embodiments, the ratio is about 1:1 to about 1:25. In some embodiments of the present invention, the pH of the coating is in the range of about pH of 1-14, preferably between 4-19 and most preferably between 5-8.

In some embodiments, the ratio of polyacrylates copolymer to the polyacrylates complex present in the coating composition ranges from about 1:1 to about 99:1, preferably about 1:1 to about 1:50 and most preferably, 1:1 to about 10:1. In some embodiments, the ratio is about 1:1 to about 1:99, preferably about 1:1 to about 1:50, and most preferably, 1:1 to about 1:10.

Another aspect of the present invention relates to a method of modulating the particle size of a substrate by applying a coating composition comprising polyacrylates copolymer or a complex thereof, wherein the porosity of the coating composition is within the range of about 1 nm to about 5 μm and the particle size is about 10 nm to about 50 μm. The particle size as contemplated in the present invention is critical for cosmetic and non-cosmetic applications. Particularly, if the substrate is a woven material, such as a woven mask, the particle size determines the type of particles that may be prevented from entry while allowing airflow through the fibers. In the case of cosmetic applications, the particle would determine leakage of the active ingredient from within the active core to the cosmetic composition.

In another aspect, the present invention also contemplates multiple coatings on a substrate such that a first coating facilitates the adhesion of a second coating onto the substrate. In some embodiments, the second coating comprises cosmetic active ingredients, pharmaceutical APIs, or compositions, including, without limiting, anti-bacterial or anti-viral agents.

### Exemplary Embodiment: Coating Composition

### A. Ammonium Acrylates Copolymer

In one embodiment, the coating composition according to the present invention comprises ammonium acrylates copolymer. Ammonium acrylates copolymer (INCI name), also known as 2-methyl-2-propenoic acid, is the ammonium salt of a polymer of two or more monomers of acrylic acid, methacrylic acid, or one of the simple esters. Ammonium acrylates copolymer is utilized in cosmetic applications as an antistatic, binder that also functions as a film former. Moreover, when utilized in a coating composition, the acrylates/ammonium acrylates copolymer also acts as an adhesive, and/or fixative.

Ammonium acrylates copolymer is commercially available as, for example, Vinysol 1086WP. In one embodiment, the ammonium acrylates copolymer is utilized in amounts ranging from about 20% to about 95% by the weight of the total coating composition, preferably at about 30% to about 95%, and more preferably at about 37% to about 50% by the weight of the total coating composition. Vinysol 1086WP has a pH of about 8, a calculated glass transition temperature, T_{g} of about 10 degree Celsius, the viscosity of about 10 mPa·s at 0.1 mm of thickness and is anionic in nature.

### Ammonium Styrene/Acrylates Copolymer

According to the embodiment, the ammonium acrylates copolymer is combined with ammonium styrene/acrylates copolymer to address the rigidity and porosity of the resultant coating composition. Ammonium styrene/acrylates copolymer is also a film former and is utilized in cosmetic compositions.

The following reaction illustrates the formation of ammonium styrene/acrylates copolymer.

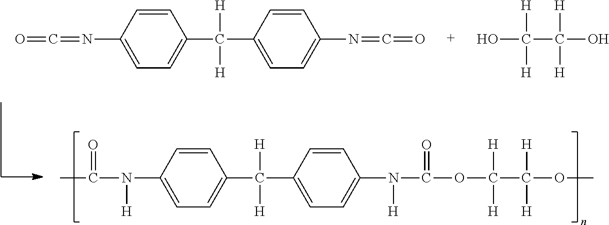

In the present invention, the amount of ammonium styrene/acrylates is in the range of about 0.1% to about 10% by the total weight of the coating composition. Ammonium styrene/acrylates copolymer is commercially available as Vinysol 1013JH. Vinysol 1013JH is reported to be anionic, with a pH between 6.5 and 9.0, and a viscosity between 5 and 500 mPa-s. The calculated glass transition temperature (T_{g}) of Vinysol 1013JH is reported to be 30° C. A higher T_{g} means that the film will be stiffer.

Vinysol 1086WP and Vinysol 1013JH are 45.0% aqueous mixtures of ammonium styrene/acrylates copolymer. Therefore, when using Vinysol 1086WP, Vinysol 1013JH, or a combination of the two, in order to achieve the concentrations of ammonium styrene/acrylates copolymer as noted above, the total concentration of Vinysol 1086WP and Vinysol 1013JH should be about 1% to about 66.7% by the total weight of the coating composition. Preferred is about 2% to 65% by the weight of the coating composition.

In some embodiments, the combined Young's modulus of Vinysol 1086WP and Vinysol 1013JH is in the range of about 1 Gpascal unit to about 20 Gpascal units. In some embodiments, the combined Young's modulus of the polyacrylates copolymer in the range of about 5 Gpascal units to about 20 GPascal units, preferably about 10 Gpascal units.

In some embodiments, the ratio of ammonium acrylates copolymer to ammonium styrene/acrylates copolymer present in the coating composition ranges from about 1:1 to about 30:1, and most preferably, 13:1 to about 15:1.

In some embodiments, the coating composition comprising the polyacrylates copolymer has a glass temperature (T_{g}) in the range from about −40 degrees Celsius to about 40 degrees Celsius. In preferred embodiments, the T_{g} is in the range of about −30 degrees Celsius to about 30 degrees Celsius.

### B. Plasticizer

The coating compositions according to the present invention are aqueous and typically comprise from about 50% to about 60% of water by weight of the total coating composition. This amount of water is from all sources, including Vinysol 1086WP and Vinysol 1013JH. However, aqueous coating compositions, as so far described, are not sprayable from a mechanical pump sprayer of the type commonly used in the cosmetic industry. At best, a narrow stream of product is produced, with little or no atomization upon striking the atmosphere. This is unacceptable for a product that is intended to cover a relatively large area with a thin film. For this reason, a third main ingredient is one or more of butylene glycol, propanediol, and glycerine. Such ingredients act as plasticizers and have multiple beneficial effects on the coating compositions. For example, these substrates increase sprayability of the wet coating composition, as well as increase flexibility. The plasticizers perform this by increasing the porosity of the coating composition. When a preferred coating composition of the invention is applied to a substrate and is allowed to dry, the dried substrate will have an average porosity between 1 nm to 5 μm, for example, 1 nm to 3 μm. Butylene glycol, propanediol, glycerine, or any combination thereof, at 0.1% to 5% can be used to achieve the pore size.

Additionally, these same plasticizers play another beneficial role. The coating compositions of the present invention should have high adhesion for the substrate material onto which it is being sprayed or applied. Sufficiently high adhesion can be ensured if the surface tension of the dried film is within 10 mN/m (milli-newtons per meter) of the surface tension of the cosmetic or skincare agent or preparation. A typical water-in-silicone or water-in-oil product has a surface tension between about 20 mN/m and 50 mN/m. Water, present in amounts from about 50% to 60% of the coating composition of the invention, has a surface tension of about 72 mN/m. Therefore, the surface tension of the coating composition of the present invention typically needs to be lowered to be within 10 mN/m of the surface tension of the substrate. In general, increasing the level of one or more of butylene glycol, propanediol, and glycerine in a composition will lower the surface tension of the composition. Advantageously, the surface tension of the coating composition can be adjusted, as needed, by the use of 0.1% to 5% butylene glycol, propanediol, glycerine, or any combination thereof. Thus, the benefits of sprayability, flexibility, comfort, and adhesion are achieved by adjusting the total concentration of any combination of these ingredients to be about 1% to 5% by weight of the total coating composition.

### C. Surfactants and Emulsifiers

One or more surfactants or emulsifiers may also be used to adjust surface tension. As noted above, coating compositions of the invention typically comprise from about 50% to about 60% of water by weight of the total coating composition. Some preferred embodiments of the present invention are single aqueous phase compositions and have little to no oil or silicone. In other preferred embodiments, the coating compositions are lightly emulsified oil-in-water emulsions. The emulsion embodiments are useful when a composition comprises fragrance oils, or when the composition will be used to deliver at least one oil-soluble actives (such as vitamin E acetate). However, one or more surfactants or emulsifiers can also be used in the present invention to adjust the surface tension of the coating compositions. In general, increasing the level of surfactant or emulsifier will lower the surface tension of the compositions. Whether used to adjust surface tension or to emulsify oil-soluble ingredients, one or more surfactants or emulsifiers should have an HLB between 8 and 12 and comprise no more than 2% of the total coating composition, typically between 0.01% to 2% of the total coating composition.

### D. Carrageenan and Hyaluronic Acid

When a coating composition of the present invention is applied to the skin and allowed to dry completely, the composition may feel tacky to the user. The tacky feel can be alleviated by the use of carrageenan and/or hyaluronic acid at concentrations of about 0.01% to about 1.0%, without interfering with the coating benefits of the composition as described herein. As an added benefit, carrageenan has a slight plasticizing effect, with the effect of reducing agglomeration and particle size. Thus, when used, carrageenan increases the sprayability of the coating composition of the invention, as well as reduces the tacky feel of the dried composition. Preferred coating compositions of the invention comprise carrageenan and/or hyaluronic acid as described.

### E. Hydrophobic Materials

Prior to applying to a surface, including keratin or non-keratin surface, the coating compositions of the present invention are in a first hydrophilic state. The ability to formulate with water-soluble ingredients in this first state is advantageous. To maintain sufficient hydrophilicity in the first state, the use of hydrophobic materials should be limited to less than about 5% based on the total weight of the coating composition, for example, 0.001% to 5%, preferably less than 2%, more preferably less than about 0.25%. Materials that are partly hydrophilic and partly hydrophobic could possibly exceed these limits, based on the performance of the final coating composition. In some embodiments of the invention, it is preferable if the coating composition comprises no hydrophobic ingredients, such as hydrophobic oils or waxes. Oils are organic substances that are liquid at ambient temperatures, such as esters, triglycerides, hydrocarbons, and silicones. A typical wax used in cosmetic compositions is carnauba wax. In some embodiments of the invention, it is most preferable if the compositions contain no hydrophobic oils or waxes.

### F. Polyurethane

Polyurethane tends to make compositions very rigid and will alter the certain minimum temperature of water required for removal of the film from the skin or hair. Therefore, the coating compositions of the invention comprise no more than 0.5%, for example, 0.0001% to 0.5%, of polyurethane.

### G. Various Ingredients

Various ingredients may be included in the coating compositions to fine-tune the consumer experience or enhance the performance of the composition. Alcohols, for example, maybe useful to speed up drying after application to the skin. Amounts of alcohol up to 5% may be useful. The coating compositions may also comprise preservatives and antioxidants, typically up to about 2% by weight of the coating composition. Thickeners, viscosity decreasing agents, and/or pH adjusters (such as caustic soda) may be used as needed to create a consumer acceptable product, typically at levels of less than 1% by weight of the coating composition. At these levels, the foregoing named ingredients do not seem to adversely affect the useful properties of the composition.

### H. Active Delivery

As noted above, the preferred coating compositions of the invention will be porous, with an average pore size of 1 nm to 5.0 μm. This pore size makes the coating compositions useful as a delivery vehicle for active ingredients, including cosmetic ingredients, anti-bacterial agents, and/or anti-viral agents. The pore size range of 1 nm to 5 μm is particularly useful for controlled entry of agents, such as anti-bacterial agents and/or anti-viral agents. Compositions of the invention may comprise antibacterial or antiviral agents. Preferably, the coating composition will comprise antibacterial or antiviral agents at about 1% to about 15% by weight of the total composition. In preferred embodiments, such agents are formulated suitably along with hydrophobic or hydrophilic carriers.

Active ingredients may be incorporated into the aqueous phase or oil phase (if there is one). Examples of hydrophilic (water-soluble) actives include: algae extract, Alpinia speciosa leaf extract, Alteromonas ferment extract, ascorbyl acid glucoside (AA2G), Citrullus lanatus (watermelon) fruit extract, Crataegus monogyna (hawthorn) flower extract, hyaluronic acid, hydrolyzed yeast protein, Lactobacillus ferment, matricaria (chamomile) extract, lens esculenta (lentil) fruit extract, Paeonia suffruticosa (peony) root extract, panthenol, pyrus malus (apple) fruit extract and Saccharum officinarum extract. Each individual hydrophilic active is typically incorporated at no more than 5.0%, for example, 0.0001% to 5%, by weight of the coating composition. Examples of hydrophobic (oil soluble) actives include Anthemis nobilis oil, bht (butylated hydroxytoluene), caffeine, Cocos nucifera (coconut) oil, salicylic acid, tetrahexyldecyl ascorbate, and tocopheryl acetate.

Each individual hydrophobic active is typically incorporated at no more than 1%, for example, 0.0001% to 1%, by weight of the coating composition. In some embodiments of the present invention, the pH of the coating is in the range of about pH of 1-14, preferably between 4-19 and most preferably between 5-8.

The following non-limiting example illustrates the present invention.

### Example. 1

| | Ingredient(s) | % Concentration (w/w) |
|---|---|---|
| Vinysol | Water | 55.88 |
| 1086WP | | |
| | Ammonium acrylates | 37.5 |
| | copolymer | |
| | Phenoxyethanol | 0.19 |
| | Sodium dehydroacetate | 0.09 |
| | Disodium EDTA | 0.09 |
| Vinysol | Water | 3.41 |
| 1013JH | | |
| | Ammonium | 2.81 |
| | styrene/acylates | |
| | copolymer | |
| | Disodium EDTA | 0.01 |
| | Phenoxyethanol | 0.01 |
| | Sodium dehydroacetate | 0.01 |

Ratio: Ammonium acrylates copolymer: Ammonium Styrene/Acrylates
Copolymer is 13.33:1
Young's modulus is 10.8 Gpa
Tg is 20.22 deg. C.
Vinysol 1086WP and Vinyso11013JH are prepared separately and mixed with each other using a prop mixer for 10 minutes in room temperature.

### Individual Core and Coating

In another aspect, the present invention provides a multiparticulated cosmetic or dermatologic composition comprising a plurality of coated active ingredients such that each individual particle of the active ingredient is coated with the coating composition described herein. Each individual particle of the active ingredient further comprises a core and a coating thereon, wherein the core is coated with the coating composition described above. In one embodiment, the cosmetic composition comprises at least one active ingredient. In other embodiments, the cosmetic composition comprises more than one active ingredient, which are either similar or different. The active ingredient may be a small molecule particle, pigment, peptide, bioparticle, chemical molecule, vitamin, antioxidant, among others. The cosmetic composition may be formulated, without limiting, in form of a cream, lotion, emulsion, dye, gel, oils, suspension, solution, powder, foam, wax, paste, foundation, soap, spray or serum.

In one embodiment of the present invention, titanium-di-oxide particles are utilized as an active ingredient. In the embodiment, each individual titanium-di-oxide particle comprises a core and a coating thereon. Accordingly, the core of individual titanium-di-oxide particle is coated with the coating composition.

The polymers used for such coating are dispersed in water prior to coating onto the core. Coating is generally achieved by spraying (i.e., a spraying step) onto each core using a microfluidic sprayer. After the spraying step, the core of the active ingredient particle is dried to ensure that all water particles present in the coated core are removed, thereby resulting in a hydrophobic complex comprising an individually coated active ingredient core. The coated core must be dried completely because the process requires a fluidic bed and forced air to levitate the active ingredients in an enclosed chamber so that the atomized sprayer would be able to deposit measurable and uniform polymeric coating. In a specific embodiment of the present invention, the core of each titanium-di-oxide particle is coated with the coating composition.

In embodiments, the thickness of the coating can be varied between 100 nm to 100 μm. In some embodiments, the thickness of the coating is in the range of about 0.5 and 3 nm. For example, in this range, titanium-di-oxide core particles exhibit sufficiently high UV absorption, reflection and scattering, thereby, making it suitable for sunscreen or sun care compositions.

In further embodiments, the coated particles comprise active ingredients in amounts ranging from about 1% to 99%, at least about 25%, 30%, 35%, 50%, 70%, 80%, 90% or 99% by the weight of the particle core. In preferred embodiments, the coated particles are present in amounts ranging from about 40-90% by the total weight of the particle core. The percentages further relate to the total weight of the cosmetic composition. Each individual coated particle comprises a core and a coating thereon.

In some embodiments, the plurality of the coated particles has less than 50% moisture content. In preferred embodiments, the plurality of coated particles has less than 10%, 5%, or 2% moisture content after drying. In preferred embodiments, the plurality of coated particles has no more than 1% moisture content and is hydrophobic.

In some embodiments, the coating composition has a specific zeta potential, prior to it being sprayed on the core of the active ingredients. The zeta potential is the measure of potential difference between the dispersion medium, i.e., coating, and the stationary layer of the particle, i.e., the core particle. In other embodiments, a specific charge is sprayed to the core of the active ingredient prior to the coating step. In some embodiments, the zeta potential is within a range of about 1000 volts to about 0.01 millivolts. In some embodiments, zeta potential ranges from about 100 volts to about 0.01 millivolts.

In specific embodiments, the polyacrylate copolymer and the complex present in the coating composition are selected based on the resultant zeta potential necessary to neutralize the charge of the core active ingredient. In this manner, subsequent to the coating step, the core particle has a resultant charge sufficient to neutralize the net charge of the cosmetic composition. In the case of titanium-di-oxide, its bandgap is 3.05 eV. After the coating is applied, the electrical potential between the coating and titanium-di-oxide will affect the adsorption of wavelength in a positive manner, thereby leading to an increase in the SPF of the cosmetic composition.

Thus, the present invention contemplates a method for regulating the charge present in the core of the active ingredient and also contemplates a method for neutralizing the charge of the active ingredient by applying a coating comprising polyacrylates copolymer or a complex thereof. This prevents free radical creation and also increases the SPF of the composition for sunscreen, sun care, make-up, or a combination thereof.

In some embodiments, the ratio of the core to the coating per particle ranges between 1:1 to about 99:1. In some embodiments, the ratio is about 1:1 to about 1:100. In some embodiments, the ratio is about 1:1 to about 1:10. In some embodiments, the ratio of the core to the coating per particle ranges from about 1:1 to about 1:99. In some embodiments, the ratio is about 1:1 to about 1:50. In some embodiments, the ratio is about 1:1 to about 1:25.

### Multiple Cores and Coating

Embodiments of the present invention further provide a multiparticulated cosmetic or dermatologic composition comprising a plurality of coated particles comprising multiple coated and/or uncoated cores.

According to an aspect of the present invention, it is contemplated that the cosmetic composition comprises a plurality of particles, wherein each particle comprises at least one active ingredient having a core and a coating thereon, and the particle cores are initially coated with a coating composition as described above. The coated particles are then brought to close proximity to a plurality of uncoated particles. Each uncoated particle includes at least one active ingredient having a core. The uncoated and coated particles are brought to close proximity by mixing together in a medium, in a uniform manner, i.e., solution, dispersion, etc. Accordingly, the present invention provides a multiparticulated cosmetic composition comprising a plurality of coated active ingredient particles having a core and a coating, and a plurality of uncoated active ingredient particles having a core, such that both coated and uncoated particles are combined at a specific ratio. The resultant multiparticulated particles are further coated with the coating composition so that the plurality of the coated active ingredient particles and the plurality of uncoated active ingredient particles exists together within the coating.

The decreased proximity (i.e., the distance between the particles) increases the efficacy of the cosmetic composition. In the absence of the coating, the active ingredient core of sunscreen agents would create free radicals that affect the efficacy of the sunscreen composition. According to the present invention, when an uncoated active ingredient particle is brought within the proximity of a coated active ingredient core particle, the uncoated core particle would neutralize any free radical that is created by the coated core particle. Moreover, any further coating of the coated core and uncoated core using polyacrylates copolymer would further reduce agglomeration and free radical creation. Such sunscreen compositions would have a higher reflective index and SPF, thereby, increased adsorption of UV rays. Further, as described above, the coating may also take into account the charge essential to neutralize the free radicals that are generated by the cores.

In a specific example of a sunscreen composition comprising zinc oxide particles, after the coating is applied, the electrical potential between the coating and zinc oxide particles will affect the adsorption wavelength in a positive manner, thereby leading to an increase in the SPF of the sunscreen or sun care composition.

In the case of a skincare agent, the multiple coatings on the combined coated and uncoated active ingredients forming a multiparticulated particle would reflect away from any blue light, which is known to irritate and affect skin layers.

In alternate embodiments, the plurality of coated core of active ingredient particles is brought to close proximity of the plurality of at least one coated active ingredient particle. In some embodiments, the coated particles are of the same active ingredient. In some embodiments, the coated articles are different particles with different core active ingredients.

The particles may be brought to close proximity in dispersion or in solution or by mixing the particles in an appropriate solvent. The weight of the coated particles is in amounts ranging from about 1% to 99%, at least about 25%, 30%, 35%, 50%, 70%, 80%, 90%, or 99% by the total weight of the particles.

The total amount of active ingredient (by weight) in the cosmetic compositions of the present invention can be about 1-100%, 10-100%, 20-100%, 20-90%, 20-80%, 20-70%, 20-60%, 20-50%, 20-40%, 20-30%, or about 20%, about 30%, about 40% about 50%, about 60%, about 70%, about 80%, about 90%, or about 100%. In one embodiment, the total amount of active ingredient is 60-90%.

In some embodiments, the coated particle has a surface tension in the range of about 10-90 mN/m. In some embodiments, the coated particles are present in an emulsion. In some embodiments, the emulsion is in oil-phase. In some other embodiments, the emulsion is in water-phase.

In specific embodiments, the coated particles are pigments or pigment powders. In some embodiments, the coated particles are skincare actives or agents. In some embodiments, the coated particles are cleansing agents. In some embodiments, the coated particles comprise pigments, skincare actives, cleansing agents, or a combination thereof.

The core of the coated particles can have any suitable particle size or shape. For example, the coated particles can be in the form of a coated powder having a particle size range of about 0.1-5000 microns or can be in the form of structures, which have a nominal particle diameter in the range of about 0.1-5000 nm.

### Exemplary Embodiments: Cosmetic Compositions

### 1. Sunscreen Composition

An exemplary embodiment of the present invention is provided. According to one aspect of the present invention, a cosmetic or dermatological composition for protecting human skin, hair or body is provided. The exemplary embodiment is a sunscreen composition directed to protecting human skin from UV radiation. The cosmetic composition comprises coated titanium-di-oxide particles, such that each individual core titanium-di-oxide particle is coated with a surface coating thereon. The cosmetic composition further comprises multi-layer coatings and, in some embodiments, the composition comprises more than one coated core particle within a multiparticulated particle.

Metal oxides such as titanium-di-oxide or zinc oxide are widely used in sunscreens. Their action is substantially based on reflection, scattering, and absorption of harmful UV radiation and largely depends on the primary particle size of the metal oxides. Titanium-di-oxide is also widely used in cosmetic formulations. Notably, metal oxides such as titanium-di-oxide or zinc oxide form reactive species, such as hydroxyl radicals due to its photocatalytic activity. Such reactive species causes agglomeration, leaching, or leakage of the active ingredient in the cosmetic composition; thus it compromises the product performance, including SPF.

Applicants of the present invention surprisingly discovered a novel multiparticulated cosmetic composition comprising a plurality of coated titanium-di-oxide particles having a core and a coating thereon, such that each individual core of the particle is coated with a coating composition described herein. Accordingly, each individual coated particle comprises a core and a coating thereon. The coating may be performed by spraying the coating composition onto the surface of the titanium-di-oxide particles via a microfluidic sprayer, such that each individual particle is coated in the process. The choice of the coating composition will depend upon the core of the active ingredient and the desired characteristics of the cosmetic composition. In this exemplary embodiment, polyacrylates copolymer and the complex thereof is utilized for coating. In preferred embodiments, ammonium acrylates copolymer and ammonium styrene acrylates copolymer are utilized as described in the exemplary embodiment above.

According to this exemplary embodiment, the core of individual titanium-di-oxide particle is coated with a liquid phase coating by admixing or spraying the coating composition to the titanium-di-oxide particles. After the core is coated, the particles are dried to remove water. In some embodiments, the particle has less than 50% moisture content upon drying. In preferred embodiments, the particle has less than 10%, 5%, 1%, or 0.1% moisture content upon drying.

In some embodiments, the coating may be chosen based on the resultant zeta potential of the core particle that is necessary to neutralize the net charge of the core upon the coating and drying process. In some embodiments, the ratio of the core to the coating ranges between 1:1 to about 99:1.

In another aspect of the exemplary embodiment, the multiparticulated coated titanium-di-oxide particles are admixed with uncoated zinc oxide particles. Upon coating the core of each of the titanium-di-oxide particle and subsequent drying, each coated core particle is admixed with a plurality of uncoated zinc oxide particles, by mixing it in a dispersion, solution, or a suspension. Thus, the resultant multiparticulated mixture comprises a plurality of coated titanium-di-oxide and uncoated zinc oxide particles. The resultant multiparticulated particles are then coated further with the same or different coating composition.

In yet another aspect of the exemplary embodiment, the multiparticulated coated titanium-di-oxide particles are admixed with coated zinc oxide particles. Upon coating the core of each titanium-di-oxide particles and subsequent drying, the coated particles are admixed with coated zinc oxide particles in dispersion, solution, or suspension. Thus, the resultant multiparticulated cosmetic composition comprises a plurality of coated titanium-di-oxide and coated zinc oxide particles. The resultant multiparticulated particles are coated further with the same or different coating composition.

In one embodiment, the weight of the coated particle is in amounts ranging from about 1% to 99%, at least about 25%, 30%, 35%, 50%, 70%, 80%, 90%, or 99% by the total weight of the particles.

In some embodiments, the ratio of the core of the coated to the uncoated particle is in the range between about 1:1 to about 99:1. In some embodiments, the ratio is about 1:1 to about 99:50. In some embodiments, the ratio is about 1:1 to about 50:1. In some embodiments, the ratio of the core to the coating ranges between about 1:1 to about 1:99. In some embodiments, the ratio is about 1:1 to about 1:50. In some embodiments, the ratio is about 1:1 to about 1:25.

In some embodiments, the ratio of the core of the coated to the coated particle is in the range between about 1:1 to about 99:1. In some embodiments, the ratio is about 1:1 to about 99:50. In some embodiments, the ratio is about 1:1 to about 50:1. In some embodiments, the ratio of the core to the coating ranges between about 1:1 to about 1:99. In some embodiments, the ratio is about 1:1 to about 1:50. In some embodiments, the ratio is about 1:1 to about 1:25.

In some embodiments of the present invention, the pH of the coating is between pH of 1-14, preferably between 4-19, and most preferably between 5-8. The total amount of active ingredient (by weight) in the cosmetic compositions of the present invention can be about 1-100%, 10-100%, 20-100%, 20-90%, 20-80%, 20-70%, 20-60%, 20-50%, 20-40%, 20-30%, or about 20%, about 30%, about 40% about 50%, about 60%, about 70%, about 80%, about 90%, or about 100%. In one embodiment, the total amount of active ingredients is 60-90%.

### 2. Make-Up Composition

This exemplary embodiment is directed to improving the appearance of the human skin by compositions comprising pigments and/or pigment powders. A cosmetic composition comprises a plurality of coated pigment particles (including powders), such that each individual pigment particle is coated with a surface coating thereon. Accordingly, each individual coated particle comprises a core and a coating thereon. The cosmetic composition further comprises muti-layer coatings and in some embodiments, the composition comprises more than one coated particle within the multiparticulated particle.

Applicants surprisingly discovered a novel cosmetic composition comprising the plurality of coated pigment particles having a core and a coating thereon, such that each individual particle is coated with a coating composition. The coating may be performed by spraying the pigment particles via a microfluidic sprayer. The coating substrate may be hydrophobic, hydrophilic or amphiphilic. The choice of the coating substrate will depend upon the core of the active ingredient and the desired cosmetic composition. For example, an amphoteric polymer may be used.

According to this exemplary embodiment, the plurality of particles comprising at least one pigment having a core is coated with a coating composition. After the pigment particles are coated, the particles are dried to remove water. In some embodiments, the pigment particle has less than 50% moisture content upon drying. In preferred embodiments, the core particles have less than 10%, 5%, 1%, or 0.1% moisture content after drying.

In another aspect of the exemplary embodiment, a plurality of multiparticulated coated particles are then admixed with uncoated zinc oxide particles in dispersion, solution or suspension. Thus, the result is a mixture of coated pigment particles and uncoated zinc oxide particles. The multiparticulated particle is then coated further with the same or different coating composition.

In yet another aspect of the exemplary embodiment, a plurality of multiparticulated coated particles are admixed with coated zinc oxide particles in dispersion, solution or suspension. Thus, the resultant is the plurality of coated pigment particles and coated zinc oxide particles. The multiparticulated particle is then further coated with the same or different coating composition.

In one embodiment of the exemplary embodiment, the weight of the coated particles is in amounts ranging from about 1% to 99%, at least about 25%, 30%, 35%, 50%, 70%, 80%, 90%, or 99% by the total weight of the particles.

### Formulation

According to an aspect of the present invention, the formulation comprising the cosmetic agent may be applied to mammalian keratinous tissue, to human skin, face or hair. The formulation comprising the cosmetic agents may be of various forms. For example, some non-limiting examples of such forms include solutions, suspensions, lotions, creams, gels, emulsions, suspension, toners, ointments, cleansing agents, exfoliating agents, liquid shampoos and hair conditioners, pastes, foams, powders, mousses, shaving creams, hydrogels, film-forming products, facial and skin masks, and the like.

Exposure to ultraviolet light may result in excessive scaling and texture changes of the stratum corneum. Therefore, the cosmetic agents of the subject invention may optionally contain a sunscreen active. As used herein, “sunscreen active” includes both sunscreen agents and physical sunblock. Suitable sunscreen actives may be organic or inorganic.

Inorganic sunscreens useful herein include the following metallic oxides: titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the cosmetic agent.

A wide variety of conventional organic sunscreen actives are suitable for use herein. Sagarin, et al., at Chapter VIII, pages 189 et seq., of Cosmetics Science and Technology (1972), discloses numerous suitable actives. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone, naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2,4,4-tetrahydroxybenzophenone, 2,2-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one), terephthalylidene dicamphor sulfonic acid and 4-isopropyl-di-benzoylmethane. Of these, 2-ethylhexyl-p-methoxycinnamate (commercially available as PARSOL MCX), 4,4-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl)aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene and mixtures of these compounds, are preferred.

More preferred organic sunscreen actives useful in the cosmetic agents useful in the subject invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, and mixtures thereof.

Also, particularly useful in the cosmetic agents are sunscreen actives such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991. The sun screening agents disclosed therein have, in a single particle, two distinct chromophore moieties which exhibit different ultra-violet radiation absorption spectra. One of the chromophore moieties absorbs predominantly in the UVB radiation range and the other absorbs strongly in the UVA radiation range.

Preferred members of this class of sun screening agents are 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N, N-(2-ethylhexyl)methyl-aminobenzoic acid ester of 2-hydroxy-4-(2-hydroxyethoxy)benzophenone; 4-N, N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N, N-di-(2-ethylhexyl)-4-aminobenzoic acid ester of 4-(2-hydroxyethoxy)dibenzoylmethane, and mixtures thereof.

Especially preferred sunscreen actives include 4,4′-t-butylmethoxydibenzoylmethane, 2-ethylhexyl-p-methoxycinnamate, phenyl benzimidazole sulfonic acid, and octocrylene. A safe and effective amount of the organic sunscreen active is used, typically from about 1% to about 20%, more typically from about 2% to about 10% by weight of the cosmetic agent. Exact amounts will vary depending upon the sunscreen or sunscreens chosen and the desired Sun Protection Factor (SPF).

In addition, the topical cosmetic agent may contain conventional cosmetic adjuvants and additives such as preservatives, antioxidants, fatty substances, oils, water, organic solvents, silicones, thickeners, emollients, emulsifiers, sunscreens, defoamers, a surfactant, a filler, a sequestering agent, an anionic, a cationic, a nonionic or an amphoteric polymer or a mixture thereof, a propellant, an acidifying agent or a basic agent, a dye, a colorant/coloring agent, an abrasive, a skin sensate, an astringent, a pigment or a nano pigment or a combustible pigment, such as for example, without limiting, iron oxides, metallic oxides or any other ingredient typically formulated in cosmetic compositions. Such cosmetic ingredients which are suitable for use in the cosmetic composition of the present invention and which are conventionally used in the skincare industry are described in, for example, the CTFA Cosmetic Ingredient Handbook, Second Edition (1992), but are not limited thereto.

The cosmetic agents of the present invention, without limiting, may be present in form of lotions, milky lotions, creams and oil, oil in emulsions, watery substances, gels, hydrogels, shampoos, hair rinses, hair conditioners, hair creams, hair dyes, hair colors, pre- or post-treatment agents for hair dyeing and coating agents for split hair, etc.

The formulation type of the cosmetic agents of the present invention may be of any type, including solution system, soluble system, emulsion system, gel system, powder dispersing system, or water-oil two-phase system.

Conventional cosmetic adjuvants which may be suitable as additives are, for example, co-emulsifiers, fats and waxes, stabilizers, thickeners, biogenic agents, film formers, fragrances, dyes, pearlescent agents, preservatives, pigments, electrolytes (for example magnesium sulphate), and pH regulators. Co-emulsifiers are preferably known W/O and also O/W emulsifiers such as polyglycerol esters, sorbitan esters, or partially esterified glycerides. Typical examples of fats are glycerides; as waxes which may be mentioned in combination with hydrophilicized growing inter alia beeswax, paraffin wax, or microcrystalline waxes.

Metal salts of fatty acids such as magnesium, aluminum and/or zinc stearate can be employed. Suitable thickeners are, for example, crosslinked polyacrylic acids and derivatives thereof, polysaccharides, more especially xanthan gum, guar-guar, agar-agar, alginate, and tyloses, carboxymethylcellulose and hydroxy ethylcellulose, and also fatty alcohols, monoglycerides and fatty acids, polyacrylates, polyvinyl alcohol, and polyvinylpyrrolidone.

Biogenic active plant extracts, protein hydrolysates, and vitamin complexes, for example, to understand. Customary film formers are, for example, hydrocolloids such as chitosan, microcrystalline chitosan or quaternized chitosan, polyvinylpyrrolidone, vinylpyrrolidone-vinyl acetate copolymers, polymers of the acrylic acid series, quaternary cellulose derivatives, and similar compounds.

Suitable preservatives are, for example, formaldehyde solution, p-hydroxybenzoate or sorbic acid. Pearlizing agents, for example, such as ethylene glycol distearic esters come coldistearate, but also fatty acids and fatty acid into consideration.

The dyes suitable for cosmetic purposes, and authorized substances may be used. Such dyes are normally used in concentrations of 0.001 to 0.1 wt %, based on the total mixture. Additional content of antioxidants is generally preferred. Thus, all suitable or customary for cosmetic and/or dermatological applications antioxidants can be used as favorable antioxidants.

The sunscreen compositions of the invention can accordingly be in liquid, paste, or solid form, for example, as a water-in-oil creams, oil-in-water creams, and lotions, aerosol foam creams, gels, oils, grease pencils, dusting powders, sprays, or hydroalcoholic lotions.

### Use

Another aspect of the present invention is the use, especially, the commercial use of a coating composition comprising polyacrylates or complex thereof, for the purpose of protecting a surface from environmental pollutants, bacterial or viral particles. In non-cosmetic embodiments, the surface may be paper, textile, metal, plastic, combustible materials, and elements, as well as clinical and hospital surfaces and related instruments.

Another aspect of the present invention is the cosmetic use of the multiparticulated cosmetic composition comprising coated and uncoated cosmetic active ingredient particles. Cosmetic uses include preventing and/or treating the signs of aging of the skin and protecting the skin from UV rays.

Other uses also include preventing and/or treating impairment in luminosity, loss of radiance of the complexion, impairment of the surface aspect of the skin, and/or impairment of the grain of the skin and/or for maintaining and/or improving the bio-mechanical properties of the skin, and/or for stimulating the energy mechanism of fibroblasts, improve hair, improve the texture of hair, improve skin radiance, protect the skin from UV radiation, act as a sunscreen, treat skin's impairments, fine lines, wrinkles, aging, or depuffing.

While particular embodiments of the present invention have been illustrated and described, it would be obvious to those skilled in the art that various other changes and modifications may be made without departing from the scope of the invention. It is therefore intended to cover in the appended claims all such changes and modifications that are within the scope of this invention.

What is claimed is:

1. A cosmetic or a dermatologic composition comprising a plurality of multiparticulated particles, wherein the multiparticulated particles comprise a plurality of coated particles, and a plurality of uncoated particles, each coated particle, and each uncoated particle comprises at least one active ingredient having a core and the plurality of coated particles further comprise a coating thereon.

2. The composition of claim 1, wherein each coated particle is coated with a coating composition.

3. The composition of claim 2, wherein the multiparticulated particles are further coated with the coating composition.

4. The composition of claim 2, wherein the coating composition comprises at least one polyacrylates copolymer in amounts of about 1-50% by weight of the total composition.

5. The composition of claim 1, wherein the active ingredient is a cosmetic agent, peptide, DNA, vitamin, organic acid, pigment, or protein.

6. The composition of claim 1, wherein the coated and uncoated cores are the same or different.

7. The composition of claim 1, wherein the coated particle is titanium-di-oxide.

8. The composition of claim 1, wherein the uncoated particle is zinc oxide.

9. The composition of claim 1, wherein the coated particle is a pigment.

10. The composition of claim 2 or 3, wherein the coating composition further comprises ammonium acrylates copolymer and ammonium styrene acrylates copolymer at a ratio of 20:1 to about 1:1.

11. A method of protecting skin or hair of a human, the method comprising applying the cosmetic composition comprising a plurality of coated particles, and a plurality of uncoated particles, each comprising at least one active ingredient having a core and a coating in an effective amount.

12. The method of claim 11, wherein the cosmetic composition is a sunscreen agent.

13. The method of claim 11, wherein the cosmetic composition is a skincare agent, a makeup agent, or a combination of both.
